(19) United States
(12) Patent Application Publication
(10) Pub. No.: US 2021/0169771 A1
(43) Pub. Date: Jun. 10, 2021

(54) BREAK-PROOF, MINERAL OIL-FREE LIPSTICK

(71) Applicant: BEIERSDORF AG, Hamburg (DE)

(72) Inventors: Franziska SCHECKER, Hamburg (DE); Merle ZEWUHN, Elmshorn (DE)

(21) Appl. No.: 16/767,536

(22) PCT Filed: Nov. 20, 2018
(86) PCT No.: PCT/EP2018/081884

§ 371 Date: May 27, 2020

(30) Foreign Application Priority Data
Dec. 1, 2017 (DE) 10 2017 221 672.7

(51) Int. Cl. A61K 8/92; A61K 8/34; A61K 8/37; A61Q 1/06

(52) U.S. Cl. CPC A61K 8/922; A61K 8/927; A61K 8/342; A61K 8/345; A61K 8/347; A61K 2800/87; A61Q 1/06; A61K 2800/43; A61K 2800/30; A61K 2800/805; A61K 8/375

(57) ABSTRACT

Lip care product containing a wax mixture of a) castor oil wax (INCI: hydrogenated castor oil), b) sunflower wax (INCI: helioanthus annals seed cera) and c) beeswax (INCI: cera alba).

The present invention relates to a lip care preparation, especially a lipstick, and to the method for producing a lipstick.

The desire to look beautiful and attractive has been rooted in people for thousands of years. Even if the ideal of beauty has undergone changes over the course of time, the desire for a flawless outward appearance has always been the aim of mankind, since a pleasant appearance increases your self-esteem and the attraction of your fellow men.

The concept of decorative cosmetics derives from the latin “decoratio”—the emphasis of beauty. In most cases, pigments are used to highlight individual parts of the body, especially the face, and to reduce irregularities in color.

In addition to face powders and rouge as pulverulent cosmetics and stick-shaped preparations, cream preparations such as day creams and cream make-up based on emulsions are used for this purpose.

The most widely used decorative cosmetics include lipstick. More than 50% of women in Germany use it. In most cases, in addition to color accentuation of the lips, it also serves as lip care.

Lipsticks generally consist of a wax matrix into which a low concentration of liquid and semi-solid oils and also pigments and fillers is incorporated. They are present in solid stick form.

A disadvantage of the prior art is the fact that lip care products, such as lip creams, lip butter etc. and especially the lipsticks of the prior art, frequently comprise mineral oils and paraffin waxes which are increasingly unpopular with consumers. In addition to a general desire for products from renewable raw materials, the concern about impurities damaging to health which could be present in the raw materials produced from crude oil also plays a role in this case. Whether this concern is actually justified can be left undecided in the context of the present disclosure. The fact is that for many consumers the desire for “mineral oil-free” products exists.

However, for the production of lipsticks in particular, the omission of mineral oils and paraffin waxes has serious disadvantages. During production, optical defects, cracks and fractures can easily occur in the stick composition during the cooling process of the molten stick composition if the temperature program for this cooling process is not adhered to exactly, for example if operated too rapidly and with an excessively high temperature difference between the melt and cooling. Moreover, if the stick composition becomes slightly too hard, which causes breaks in the solid stick composition during demolding (filling) from (in) the stick sleeve, or too soft, so that residues remain in the stick mold (casting annulus) when the stick composition is demolded which must first be cleaned again in a complex cleaning process before the next filling. The variations in quality of substitutes of natural origin are the other factors which reinforce these negative effects. Overall, the replacement of mineral oil products results in a significant increase in unsellable rejects.

The object of the present invention, therefore, was to avoid the disadvantages of the prior art and to develop a lip care product that is free of mineral oil products which at the same time can be produced more easily and with fewer rejects and also at higher quality.

In particular, mineral oil-free lip care products (especially lipsticks) of the prior art also have the disadvantage that they are not particularly stable on storage, but are relatively highly temperature and light sensitive, i.e. in the case of temperature fluctuations or light they become relatively slightly inhomogeneous and brittle/cracked. Mineral oil-free sticks of the prior art also have the disadvantage that they exhibit worse glide characteristics on the lips compared to mineral oil-containing sticks. These sticks feel waxy/sticky/unpleasant.

Therefore, the object of the present invention was also to reduce these deficiencies of the prior art.

Surprisingly, the objects are achieved by a lip care product, comprising a wax mixture of

a) castor wax (INCI: Hydrogenated Castor Oil),

b) sunflower wax (INCI: Helianthus Annuus Seed Cera) and

c) beeswax (INCI: Cera Alba).

The lip care preparation, i.e. the lip care product, is also referred to as a preparation in the context of the present disclosure.

In this case, it is preferred in accordance with the invention if the preparation of the lip care preparation is free of mineral oil, paraffin wax, microcrystalline wax, shellac wax and polyethylene waxes.

Advantageous embodiments of the present invention are characterized in that the preparation comprises castor wax at a concentration of 2 to 10% by weight, based on the total weight of the preparation, wherein a concentration range of 3 to 4% by weight, based on the total weight of the preparation, is preferred in accordance with the invention. In contrast, above 10% by weight, the stick becomes too waxy-soft and unstable.

In addition, it is advantageous in accordance with the invention if the preparation comprises sunflower wax at a concentration of 2 to 10% by weight, based on the total weight of the preparation. Here, the preferred use concentration in accordance with the invention is 3 to 5% by weight, based on the total weight of the preparation. In contrast, above 10% by weight in lipsticks, the stick composition becomes so hard that it can no longer be reasonably applied to the skin.

Furthermore, the lip care products according to the invention are characterized in that the preparation comprises beeswax at a concentration of 5 to 20% by weight and preferably 10 to 15% by weight, based on the total weight of the preparation.

The advantageous embodiments of the present invention in accordance with the invention are also characterized in that the preparation comprises sunflower wax and castor wax in a ratio by weight from 1:1 to 4:1.

In addition to the wax components, a lip care product according to the invention advantageously comprises 30 to 60% by weight of oils such as Guerbet alcohols and/or triglycerides. In accordance with the invention, these oils are preferably selected from the group of the compounds octyldodecanol, castor oil (INCI: Ricinus Communis Seed Oil), cocoglycerides (INCI: Coco Glycerides), jojoba oil (INCI: Simmondsia Chinensis Seed Oil).

Furthermore, advantageous lip care products according to the invention are characterized in that the preparation comprises cetyl palmitate, shea butter and/or cetearyl alcohol.

The advantageous use concentration according to the invention for shea butter is 0.5 to 6% by weight, based on the total weight of the preparation.

The advantageous use concentration according to the invention for cetyl palmitate is 2 to 10% by weight, based on the total weight of the preparation.

The advantageous use concentration according to the invention for cetearyl alcohol is 5 to 15% by weight, based on the total weight of the preparation.

In addition, it is advantageous in accordance with the invention if the lip care preparation comprises butylhydroxytoluene (BHT). The use concentration according to the invention for this substance is from 0.01 to 0.05% by weight, based on the total weight of the preparation.

The lip care products according to the invention may contain up to 2% by weight, based on the total weight of the preparation, of water and/or hydrophilic constituents such as glycerol, panthenol etc.

For certain purposes, it can be of advantage in accordance with the invention if the preparation contains UV filters, pigments, perfumes and/or aroma substances.

If the lip care product according to the invention contains pigment(s), it is advantageous in accordance with the invention if the pigment(s) are selected from compounds with color indices CI 77891, CI 77492, CI 15850.

If the preparation according to the invention contains UV filters, it is advantageous in accordance with the invention if the UV filters are selected from the group of the compounds octocrylene, homosalate, ethylhexyl salicylate, ethylhexyl methoxycinnamate and butylmethoxy dibenzoylmethane.

Last but not least, advantageous embodiments of the present invention in accordance with the invention are characterized in that the preparation contains bis-diglyceryl polyacyladipate-2. This compound may be used advantageously in accordance with the invention at a concentration of 5 to 15% by weight, based on the total weight of the preparation.

In accordance with the invention, the preparation according to the invention, i.e. the lip care product according to the invention, is preferably in the form of a lipstick. This means that the lip care product in accordance with the invention is preferably a lipstick.

Also in accordance with the invention is the method for producing this lipstick, which is characterized in that

the formulation constituents are firstly melted and homogeneously mixed in a boiler,

the preparation is subsequently poured into a casting annulus at a temperature of 5-15° C. above its solidification point,

then cooled at a cooling temperature of +6° C. to +25° C.

and the solidified lipstick shaped composition is expelled from the casting annulus and is inserted into a lipstick container,

and also

a lipstick which has been produced by this method.

### EXAMPLE

The following example is intended to illustrate the present invention without limiting it. All quantities, proportions and percentages, unless stated otherwise, are based on the weight and the total amount or the total weight of the preparations.

| | | |
|---|---|---|
| Phase A | Castor wax | 4% |
| | Sunflower wax | 5% |
| | Beeswax | 10% |
| Phase B | Octyldodecanol | 30% |
| | Cocoglycerides | 10% |
| | Butyrospermum parkii butter | 5% |
| | UV filter | to |
| | | 100 |
| Phase C | Ricinus communis seed oil | 20% |
| | Pigment | to |
| | | 100 |
| Phase D | Aroma | to |
| | | 100 |

Bulk Preparation

Phase A: melt wax

Phase B: add to phase A

Phase C: disperse pigment in castor oil, add to phase A+B

Heat to at least 85° C. so that everything is dissolved/melted

Add phase D

Filling
cool composition (bulk) to casting temperature (70° C.). The solidification point of the composition is ca. 60° C.
fill into the casting annulus with the aid of the filling machine. Casting annulus temperature: 12° C.
mechanically demold the sticks

Prior to (renewed) filling, the casting annulus is heated to 17° C.

1.-16. (canceled)

17. A lip care product, wherein the product comprises a mixture of (a) castor wax, (b) sunflower wax and (c) beeswax.

18. The lip care product of claim 17, wherein the product is free of mineral oil, paraffin wax, microcrystalline wax, shellac wax and polyethylene waxes.

19. The lip care product of claim 17, wherein the product comprises from 2% to 10% by weight of (a), based on a total weight of the product.

20. The lip care product of claim 17, wherein the product comprises from 2% to 10% by weight of (b), based on a total weight of the product.

21. The lip care product of claim 17, wherein the product comprises from 5% to 20% by weight of (c), based on a total weight of the product.

22. The lip care product of claim 17, wherein the product comprises, based on a total weight of the product, from 2% to 10% by weight of (a), from 2% to 10% by weight of (b) and from 5% to 20% by weight of (c).

23. The lip care product of claim 22, wherein the product comprises from 3% to 4% by weight of (a), from 3% to 5% by weight of (b) and from 10% to 15% by weight of (c).

24. The lip care product of claim 17, wherein the product comprises sunflower wax and castor wax in a ratio by weight of from 1:1 to 4:1.

25. The lip care product of claim 22, wherein the product comprises sunflower wax and castor wax in a ratio by weight of from 1:1 to 4:1.

26. The lip care product of claim 17, wherein the product further comprises, based on a total weight of the product, from 30% to 60% by weight of one or more of octyldodecanol, castor oil, cocoglycerides, jojoba oil.

27. The lip care product of claim 23, wherein the product further comprises, based on a total weight of the product, from 30% to 60% by weight of one or more of octyldodecanol, castor oil, cocoglycerides, jojoba oil.

28. The lip care product of claim 17, wherein the product further comprises one or more of cetyl palmitate, shea butter, cetearyl alcohol.

29. The lip care product of claim 17, wherein the product further comprises butylhydroxytoluene (BHT).

30. The lip care product of claim 17, wherein the product further comprises one or more of UV filters, pigments, perfumes, aroma substances.

31. The lip care product of claim 17, wherein the product further comprises one or more pigments selected from compounds with color indices CI 77891, CI 77492, CI 15850.

32. The lip care product of claim 17, wherein the product further comprises one or more of octocrylene, homosalate, ethylhexyl salicylate, ethylhexyl methoxycinnamate, butylmethoxy dibenzoylmethane.

33. The lip care product of claim 17, wherein the product further comprises bis-diglyceryl polyacyladipate-2.

34. The lip care product of claim 26, wherein the product further comprises one or more of cetyl palmitate, shea butter, cetearyl alcohol.

35. The lip care product of claim 17, wherein the product is present in the form of a lipstick.

36. A method of making the lipstick of claim 35, wherein the method comprises (i) melting all constituents of the lipstick in a boiler, (ii) pouring a resultant melt into a casting annulus at a temperature of 5-15° C. above a solidification point of the melt, (iii) cooling the melt to a temperature of from +6° C. to +25° C. to solidify the melt and (iv) expelling the solidified lipstick shaped melt from the casting annulus and inserted it into a lipstick container.
